SEQUENCE LISTING (3 sequences)

SEQ ID NO: 1 - protein, 1404 aa, homo sapiens
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIWKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTAPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTSDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGEMTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 2 - DNA, 5042 bp, homo sapiens
gcggccgcgactattcggtacctgaaaacaacgatggcatggaaaacacttcccatttacctgttgttgctgctgtctgttttcgtgattcagcaagtttcatctcaagatttatcaagctgtgcagggagatgtggggaagggtattctagagatgccacctgcaactgtgattataactgtcaacactacatggagtgctgccctgatttcaagagagtctgcactgcggagctttcctgtaaaggccgctgctttgagtccttcgagagagggagggagtgtgactgcgacgcccaatgtaagaagtatgacaagtgctgtcccgattatgagagtttctgtgcagaagtgcataatcccacatcaccaccatcttcaaagaaagcacctccaccttcaggagcatctcaaaccatcaaatcaacaaccaaacgttcacccaaaccaccaaacaagaagaagactaagaaagttatagaatcagaggaaataacagaagaacattctgtttctgaaaatcaagagtcctcctcctcctcctcctcttcctcttcttcttcaacaatttggaaaatcaagttttccaaaaattcagctgctaatagagaattacagaagaaactcaaagtaaaagataacaagaagaacagaactaaaaagaaacctacccccaaaccaccagttgtagatgaagctggaagtggattggacaatggtgacttcaaggtcacaactcctgacacgtctaccacccaacacaataaagtcagcacatctcccaagatcacaacagcaaaaccaataaatcccagacccagtcttccacctaattctgatacatctaaagagacgtctttgacagtgaataaagagacaacagttgaaactaaagaaactactacaacaaataaacagacttcaactgatggaaaagagaagactacttccgctaaagagacacaaagtatagagaaaacatctgctaaagatttagcacccacatctaaagtgctggctaaacctacacccaaagctgaaactacaaccaaaggccctgctctcaccactcccaaggagcccacgcccaccactcccaaggagcctgcatctaccacacccaaagagcccacacctaccaccatcaagtctgcacccaccacccccaaggagcctgcacccaccaccaccaagtctgcacccaccactcccaaggagcctgaacccaccaccaccaaggagcctgcacccaccactcccaaggagcctgcacccaccaccaccaaggagcctgcacccaccaccaccaagtctgcacccaccactcccaaggagcctgcacccaccacccccaagaagcctgccccaactacccccaaggagcctgcacccaccactcccaaggagcgcacacccaccactcccaaggagcctgcacccaccaccaaggagcctgcacccaccactcccaaagagcttgcacccactgcccccaagaagcctgccccaactacccccaaggagcctgcacccaccactcccaaggagcctgcacccaccaccaccaaggagccttcacccaccactcccaaggagcctgcacccaccaccaccaagctcgcacccaccactaccaaggagcctgcacccaccactaccaagtctgcacccaccactcccaaggagccttcacccaccaccaccaaggagcctgcacccaccactcccaaggagcctgcacccaccacccccaagaagcctgccccaactacccccaaggagcctgcacccaccactcccaaggaacctgcacccaccaccaccaagaagcctgcagccaccgctcccaaagagcctgccccaactacccccaaggagactgcacccaccacccccaagaagctcacgcccaccacccccgagaagctcgcacccaccacccctgagaagcccgcacccaccacccctgaggagctcgcacccaccacccctgaggagcccacacccaccacccctgaggagcctgctcccaccactcccaaggcagcggctcccaacacccctaaggagcctgctccaactacccctaaggagcctgctccaactacccctaaggagcctgctccaactacccctaaggagactgctccaactacccctaaagggactgctccaactaccctcaaggaacctgcacccactactcccaagaagcctgcctccaaggagcttgcacccaccaccaccaaggagcccacatccaccacctctgacaagcccgctccaactacccctaaggggactgctccaactacccctaadgadcctgctccaactacccctaaggagcctgctccaactacccctaaggggactgctccaactaccctcaaggaacctgcacccactactcccaagaagcctgcccccaaggagcttgcacccaccaccaccaaggggcccacatccaccacctctgacaagcctgctccaactacacctaaggagactgctccaactacccccaaggagcctgcacccactacccccaagaagcctgctccaactactcctgagacacctcctccaaccacttcagaggtctctactccaactaccaccaaggagcctaccactatccacaaaagccctgatgaatcaactcctgagctttctgcagaacccacaccaaaagctcttgaaaacagtcccaaggaacctggtgtacctacaactaagactcctgcagcgactaaacctgaaatgactacaacagctaaagacaagacaacagaaagagacttacgtactacacctgaaactacaactgctgcacctaagatgacaaaagagacagcaactacaacagaaaaaactaccgaatccaaaataacagctacaaccacacaagtaacatctaccacaactcaagataccacaccattcaaaattactactcttaaaacaactactcttgcacccaaagtaactacaacaaaaaagacaattactaccactgagattatgaacaaacctgaagaaacagctaaaccaaaagacagagctactaattctaaagcgacaactcctaaacctcaaaagccaaccaaagcacccaaaaaacccacttctaccaaaaagccaaaaacaatgctcagagtgagaaaaccaaagacgacaccaactccccgcaagatgacatcaacaatgccagaattgaaaccctacctcaagaatagcagaagccatgctccaaaccaccaccagacctaaccaaactccaaactccaaactagttgaagtaaatccaaagagtgaagatgcaggtggtgctgaaggagaaacacctcatatgcttctcaggccccatgtgttcatgcctgaagttactcccgacatggattacttaccgagagtacccaatcaaggcattatcatcaatcccatgctttccgatgagaccaatatatgccatggtaagccagtagatggactgactactttgcgcaatgggacattagttgcattccgaggtcattatttctggatgctaagtccattcagtccaccatctccagctcgcagaattactgaagtttggggtattccttcccccattgatactgtttttactaggtgcaactgtgaaggaaaaactttcttctttaaggattctcagtactggcgttttaccaatgatataaaagatgcagggtaccccaaaccaattttcaaaggatttggaggactaactggacaaatagtggcagcgctttcaacagctaaatataagaactggcctgaatctgtgtattttttcaagagaggtggcagcattcagcagtatatttataaacaggaacctgtacagaagtgccctggaagaaggcctgctctaaattatccagtgtatggagaaatgacacaggttaggagacgtcgctttgaacgtgctataggaccttctcaaacacacaccatcagaattcaatattcacctgccagactggcttatcaagacaaaggtgtccttcataatgaagttaaagtgagtatactgtggagaggacttccaaatgtggttacctcagctatatcactgcccaacatcagaaaacctgacggctatgattactatgccttttctaaagatcaatactataacattgatgtgcctagtagaacagcaagagcaattactactcgttctgggcagaccttatccaaagtctggtacaactgtccttagactgatgagcaaaggaggagtcaactaatgaagaaatgaataataaattttgacactgaaaaacattttattaataaagaatattgacatgagtataccagtttatatataaaaatgtttttaaacttgacaatcattacactaaaacagatttgataatcttattcacagttgttattgtttacagaccatttaattaatatttcctctgtttattcctcctctccctcccattgcatggctcacacctgtaaaagaaaaaagaatcaaattgaatatatcttttaagaattcaaaactagtgtattcacttaccctagttcattataaaaaatatctaggcattgtggatataaaactgttgggtattctacaacttcaatggaaattattacaagcagattaatccctctttttgtgacacaagtacaatctaaaagttatattggaaaacatggaaatattaaaattttacacttttactagctaaaacataatcacaaagctttatcgtgttgtataaaaaaattaacaatataatggcaataggtagagatacaacaaatgaatataacactataacacttcatattttccaaatcttaatttggatttaaggaagaaatcaataaatataaaatataagcacatatttattatatatctaaggtatacaaatctgtctacatgaagtttacagattggtaaatatcatctgctcaacatgtaattatttaataaaactttggaacattaaaaaaataaattggaggcttaaaaaaaaaaaaaaaaa

SEQ ID NO: 3 - protein, 7 aa, Artificial sequence
  Chemically synthesized sequence
KEPAPTT

(19) United States
(12) Patent Application Publication
(10) Pub. No.: US 2009/0104148 A1
(43) Pub. Date: Apr. 23, 2009

(54) Treatment and prevention of joint disease

(76) Inventors: Gregory D. Jay, Norfolk, MA (US); Khaled A. Elsaid, Kingston, RI (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/284,025

(22) Filed: Sep. 18, 2008

Related U.S. Application Data
(60) Provisional application No. 60/994,372, filed on Sep. 18, 2007.

(51) Int. Cl. A61K 38/20; A61K 39/395; A61K 31/711; A61P 19/02

(52) U.S. Cl. 424/85.2; 424/130.1; 514/44

(57) ABSTRACT

The invention provides compositions and methods for preserving, prolonging, or augmenting joint lubrication by contacting a tissue of a joint such as a knee, elbow, or other articulating joint, with a tribonectin and an inhibitor of a proinflammatory mediator.

### RELATED APPLICATIONS

This patent application claims the benefit of U.S. Provisional Patent Application Ser. No. 60/994,372, filed Sep. 18, 2007, the contents of which are herein incorporated by reference in their entirety.

### FIELD OF THE INVENTION

The present invention relates to the lubrication of mammalian joints.

### BACKGROUND OF THE INVENTION

Osteoarthritis (OA) is a common form of joint disease. Factors which contribute to the development of OA include a family history of OA, previous damage to the joint through injury or surgery, and age of the joint, i.e., “wear and tear” of the articulating surfaces of the joint. OA is very common in older age groups, but can affect children as well.

Current treatment is directed to relieving pain and other symptoms of OA, e.g., by administering analgesics and anti-inflammatory drugs. Other therapeutic approaches include viscosupplementation by administering hyaluronic acid and derivatives thereof to joint tissue to increase the viscosity of synovial fluid.

### SUMMARY OF THE INVENTION

The invention provides a method for preserving or augmenting joint lubrication by contacting a tissue of a joint such as a knee, elbow, or other articulating joint, with a tribonectin and an inhibitor of a proinflammatory mediator. For example, the mediator is a cytokine such as interleukin-1β (IL-1β) or tumor necrosis factor-α (TNF-α). Alternatively the mediator is another proinflammatory mediator such as a cathepsin, e.g. cathepsin B. Preferably, the combination leads to a synergistic effect in preserving or augmenting joint lubrication. The inhibitor reduces the expression or function of TNF-α such as an antibody or antibody conjugate that binds to TNF-α Exemplary compositions include ENBREL™. In another example, the inhibitor reduces the expression or function of a proinflammatory IL-1, such as an IL-1 RA, e.g., KINERET™. By reducing inflammation, the methods also prolong joint lubrication, e.g., augmented joint lubrication after administration of a lubricating compound to the joint, by reducing catabolism of endogenously produced and exogenous administered lubricating compositions.

As an alternative to the combination therapy approach described above, one or more inhibitors that reduce the expression or function of TNF-α are administered alone either subcutaneously at or near an articulating joint or intra-articularly directly to the joint itself.

The compounds are administered directly to or adjacent to a target joint. For example, the inhibitor is administered (injected or infused) directly into a joint. Alternatively, inhibitor is administered subcutaneously, e.g., into cutaneous tissue adjacent to the target joint tissue of the affected joint. For example, the compounds are injected subcutaneously 5 mm deep (shallow injection just beneath the skin surface) to 1.5 cm deep into the tissue (i.e., approaching or entering the joint) of an adult human. The compounds are injected 3-4 mm deep into the tissue (shallow injection) to approximately 10 mm deep for a child. The tribonectin and inhibitor are administered simultaneously or sequentially. In some cases, the compositions are compounded together for simultaneously delivery. Optionally hyaluronic acid is also administered. Accordingly, a composition containing a tribonectin and an inhibitor of a proinflammatory mediator is within the invention.

The compositions are administered in the following dose ranges: tribonectin or recombinant lubricin (0.1 μg/ml-1 mg/ml); HA (0.1 mg/ml-50.0 mg/ml); inhibitor of TNF (0.1-10 mg/kg). For example, a TNF inhibitor is administered at 0.5-5 mg/kg. The compositions are administered to a joint in a volume of 0.5-5 ml. For example, the compositions are administered to a joint, e.g., a knee, shoulder, or elbow, in a volume of approximately 1 ml. In the event of a acute injury, which may be characterized by swelling or effusion, an abnormal amount of fluid is present in the joint. In the latter case, up to 5 ml of a solution is administered to the joint. For example, the joint is first aspirated to remove the effusion and then the therapeutic composition containing a lubricating agent and an anti-inflammatory agent is inject or infused into the joint.

The compositions described herein are purified. For example, tribonectin polypeptides are biochemically purified. The enzyme chymotrypsin cleaves at sites which bracket amino acids encoded by exon 6 (underlined in SEQ ID NO:1) of the MSF gene. Thus, a polypeptide containing amino acids encoded by exon 6 of the MSF gene (but not any other MSF exons) is prepared from a naturally-occurring or recombinantly produced MSF gene product by enzymatic digestion with chymotrypsin. The polypeptide is then subjected to standard biochemical purification methods to yield a substantially pure polypeptide suitable for therapeutic administration, evaluation of lubricating activity, or antibody production. Alternatively, the compositions are synthetically or recombinantly produced.

Therapeutic compositions are administered in a pharmaceutically acceptable carrier (e.g., physiological saline). Carriers are selected on the basis of mode and route of administration and standard pharmaceutical practice. A therapeutically effective amount of a therapeutic composition (e.g., lubricating polypeptide, anti inflammatory compound, or other composition) is an amount which is capable of producing a medically desirable result, e.g., boundary lubrication of a mammalian joint, in a treated animal.

The combination therapy described herein confers a clinical benefit such as increased lubrication, reduced inflammation/swelling, increased range of motion/mobility, and/or decrease in pain. A medically desirable result is a reduction in pain (measured, e.g., using a visual analog pain scale described in Peyron et al., 1993, J. Rheumatol.(suppl.39):10-15) or increased ability to move the joint (measured, e.g., using pedometry as described in Belcher et 30 al., 1997, J. Orthop. Trauma 11: 106-109). Lubricity (μ) of synovial fluid is measured before or after treatment by reaspirating a small volume of synovial fluid from the affected joint and testing the lubricating properties in vitro using know methods, e.g., a friction apparatus described in Jay et al., 1992, Conn. Tiss. Res. 28:71-88 or Jay et al., 1998, J. Biomed. Mater. Res. 40:414-418 and U.S. Pat. No. 6, 690,562.

As is well known in the medical arts, dosage for any one animal depends on many factors, including the animal's size, body surface area, age, the particular compound to be administered, sex, time and route of administration, general health, and other drugs being administered concurrently. Subjects to be treated include humans, companion animals such as dogs, cats as well as horses, oxen, donkey, cow, sheep, pig, rabbit, monkey or mouse. Administration is generally local to an injured or inflamed joint. Alternatively, the compositions are administered subcutaneously in close proximity to the joint or via a timed-release implant placed in close proximity to a joint for slow release at the site of an injured or inflamed joint. Optionally, hyaluronic acid (HA) is co-administered. Administration of a tribonectin and an inhibitor of an anti-inflammatory mediator leads to a synergistic clinical benefit.

A purified composition such as a protein or peptide (e.g., antibody, fusion protein) is at least 60%, by weight, free from proteins and naturally occurring organic molecules with which it is naturally associated. Preferably, the preparation is at least 75%, more preferably 90%, and most preferably at least 99%, by weight, the desired composition. A purified antibody may be obtained, for example, by affinity chromatography. By “substantially pure” is meant a nucleic acid, polypeptide, or other molecule that has been separated from the components that naturally accompany it. Typically, the polypeptide is substantially pure when it is at least 60%, 70%, 80%, 90%, 95%, or even 99%, by weight, free from the proteins and naturally-occurring organic molecules with which it is naturally associated. For example, a substantially pure polypeptide may be obtained by extraction from a natural source, by expression of a recombinant nucleic acid in a cell that does not normally express that protein, or by chemical synthesis.

By “substantially identical,” when referring to a protein or polypeptide, is meant a protein or polypeptide exhibiting at least 75%, but preferably 85%, more preferably 90%, most preferably 95%, or even 99% identity to a reference amino acid sequence. For proteins or polypeptides, the length of comparison sequences will generally be at least 20 amino acids, preferably at least 30 amino acids, more preferably at least 40 amino acids, and most preferably 50 amino acids or the full length protein or polypeptide. Nucleic acids that encode such “substantially identical” proteins or polypeptides constitute an example of “substantially identical” nucleic acids; it is recognized that the nucleic acids include any sequence, due to the degeneracy of the genetic code, that encodes those proteins or polypeptides. In addition, a “substantially identical” nucleic acid sequence also includes a polynucleotide that hybridizes to a reference nucleic acid molecule under high stringency conditions.

By “high stringency conditions” is meant any set of conditions that are characterized by high temperature and low ionic strength and allow hybridization comparable with those resulting from the use of a DNA probe of at least 40 nucleotides in length, in a buffer containing 0.5 M NaHPO4, pH 7.2, 7% SDS, 1 mM EDTA, and 1% BSA (Fraction V), at a temperature of 65 oC, or a buffer containing 48% formamide, 4.8XSSC, 0.2 M Tris-Cl, pH 7.6, 1X Denhardt's solution, 10% dextran sulfate, and 0.1% SDS, at a temperature of 42 oC. Other conditions for high stringency hybridization, such as for PCR, Northern, Southern, or in situ hybridization, DNA sequencing, etc., are well known by those skilled in the art of molecular biology. See, e.g., F. Ausubel et al., Current Protocols in Molecular Biology, John Wiley & Sons, New York, N.Y., 1998, hereby incorporated by reference.

The term “isolated DNA” is meant DNA that is free of the genes which, in the naturally occurring genome of the organism from which the given DNA is derived, flank the DNA. Thus, the term “isolated DNA” encompasses, for example, cDNA, cloned genomic DNA, and synthetic DNA.

Other features and advantages of the invention will be apparent from the following description of the preferred embodiments thereof, and from the claims. Publications, U.S. patents and applications, GenBank/NCBI accession numbers, and all other references cited herein, are herby incorporated by reference.

### BRIEF DESCRIPTION OF THE DRAWINGS

FIG. 1 is a photograph of a histologically stained rat articular cartilage using mab S6.89. A-A representative right knee joint one week following the initiation of the arthritic model showing less lubricin staining in the superficial zone articular chondrocytes. B-A representative contra-lateral left knee joint one week following the initiation of the arthritic model showing presence of lubricin.

FIG. 2 is a bar graph showing ex-vivo μ of ACL transected (ACLT) (n=6) and contralateral (CL) (n=6) rat joints at 1 and 4 weeks following transection. *Indicates that μ values of ACLT joints at week 4 were significantly (p=0.02) higher than μ values of CL joints at week 4 and higher (p=0.03) than μ values of ACLT joints at week 1.

FIG. 3 is is a bar graph showing SF lavage sGAG levels of ACL transected (ACLT) (n=6) and contralateral (CL) (n=6) rat joints at 1 and 4 weeks following transaction. * Indicates that sGAG levels of ACLT joints at week 1 and 4 were significantly (p<0.001) higher than sGAG levels of CL joints at week 1 and 4.

FIG. 4 is a bar graph showing Lubricin synovial fluid (SF) lavage levels in ACL-transected (ACLT), contralateral (CL), sham and contralateral sham (sham-CL) following etanercept-treatment (n=5), no treatment(n=5) or sham surgery (n=4) at 2 weeks following ACLT. *Indicates that lubricin SF lavage levels were significantly (p<0.001) higher in the etanercept-treated ACLT joints compared to non-treated ACLT joints.

FIG. 5 is a bar graph showing ex-vivo joint coefficient of friction (μ) of ACL-transected (ACLT), contralateral (CL), sham and contralateral sham (sham-CL) following etanercept-treatment (n=5), no treatment (n=5) or sham surgery (n=4) at 2 weeks following ACLT. *Indicates that μ values of etanercept-treated ACLT joints were significantly (p=0.021) lower than the non-treated ACLT joints.

FIG. 6 is a bar graph showing sGAG SF lavage levels in ACL-transected (ACLT), and contralateral (CL) joints following etanercept-treatment (n=5), or no treatment (n=5) at 2 weeks following ACLT. *Indicates that sGAG SF lavage levels were significantly (p<0.001) lower in the etanercept-treated joints compared to non-treated joints.

FIG. 7 is a bar graph showing synovial fluid (SF) lavage lubricin levels in ACL-transected (ACLT) and contralateral (CL) joints of animals with ACLT, Etanercept-A, Etanercept-B treatments and sham surgery. *Indicates that SF lavage lubricin levels in treatment-A were significantly (p<0.01) higher than SF lavage lubricin levels in ACLT and treatment-B. Statistical analyses were performed by student's t-test.

FIG. 8 is a bar graph showing percentage surface coverage of articular surface by lubricin ACL-transected (ACLT) and contra lateral (CL) joints of animals with ACLT, Etanercept-A, Etanercept-B treatments and sham surgery. *Indicated that percentage surface coverage of articular surface by lubricin in the ACLT joints was significantly (p<0.01) lower than in treatment-A, treatment-B or sham surgery.

### DETAILED DESCRIPTION

Endogeneous tribosupplementation includes a role for anti-inflammatory agents such as TNF-α blockers such as Etanercept or IL-1 inhibitors in the treatment of injured joints or joints that are at risk of degeneration following joint trauma.

### Joint Injury and Degeneration of Joint Tissue

Post-traumatic causes of OA involve catabolic cytokines. IL-1 has been shown to down regulate lubricin, which by itself does not lead to OA but may play a degenerative role since IL-1β has also been shown to result in less lubricin secretion in vitro. The SF levels of IL-1β and TNF-α are elevated following an acute ACL-injury, and remain elevated 3 weeks post injury. Both IL-1β and TNF-α stimulate the secretion of latent cysteine proteinases, primarily that of cathepsin B, from synovial fibroblasts. Cathepsin B has been shown to possess a strong ability to proteolytically degrade lubricin and abolish lubricin's lubricating ability. Inhibition prolongs and augments joint lubrication.

ACL injury has both immediate and long-term implications on patients' quality of life, long-term disability and risk for OA. Many ACL-injured subjects suffer a severe functional deficit. Although one third of them may improve function with rehabilitation alone, there is substantial evidence that they will manifest articular cartilage degeneration. On average, patients with OA following ACL injury are 15- to 20-years younger than those with primary OA when they seek medical help for their symptoms. Reasons for this profile include the initial inflammatory response, the presence of concomitant injuries, altered kinematics, abnormal contact stresses, and/or the increased incidence of “giving way” following injury producing additional insult to the cartilage and menisci. All of these factors may disrupt the homeostasis of the joint and place the knee at risk for early OA. Therefore, surgeons advocate reconstruction of the ACL to decrease joint laxity, to restore an active lifestyle, and to reduce future knee injuries. The impact of a non-reconstructed ACL injury upon the chondroprotective features of the joint has not been established. The inflammatory response at the time of injury decreases the lubricating ability of the joint therefore sets the stage for post-traumatic arthritis.

Patients who undergo ACL reconstruction also exhibit progressive articular cartilage damage in the reconstructed knee. In a recent 14-year follow-up study of 205 male atheletes, 78% had degenerative signs in their injured knee compared to 4% in their uninjured knee (Von Porat et al., 20054, Ann. Rheum. Dis. 63:269-273). In a 5-year follow-up, others (Daniel et al., 1994, Am. J. Sports Med.22:632-644) determined that both acute and chronic ACL reconstructed knees had significantly great radiographic evidence of OA as compared to those in a conservatively treated (non-operative) group. These findings were confirmed by another group (Fithian et al., 2002, Orthoped. Clin. N. America 33:621-636) who found that the incidence of chondropathy was 94% and 92% with and without meniscus tears respectively using MRI. Although radiographic signs of OA were present within five years of surgery, the mechanisms of chondropathy were heretofore unknown.

Studies were carried out to evaluate the association of dysfunction of joint tribologic properties with acute ACL injuries in both an animal model and clinical aspirates. Inhibiting inflammation was found to preserve lubricating properties and prevent chondropathy.

### Lubrication of Articulating Joints

Re-establishing the normal lubricating ability of the diarthrodial joint is important following an injury. Catabolism of lubricin eliminates the lubricating ability of synovial fluid. This process occurs both within the synovial fluid as well as the lubricin located on the surface of articular cartilage. Introducing lubricin and hyaluronic acid together (i.e., artificial synovial fluid) is a potential therapy but the half life of this therapy may be short. Combination therapy in which anti-inflammatory treatment is administered simultaneously or sequentially preserves the introduced lubricant and prevent its digestion by activated proteases. Intervention of the inflammatory process is accomplished at multiple stages in the cyclo-oxygenase and lipoxy-oxygenase inflammatory cascades. One method involves treating the injured diarthrodial joint at the same time through the intra-articular administration of IL-1 inhibitors and TNF-beta inhibitors to block the upstream proinflammatory cytokines, which initiate the downward cascade resulting in protease expression. The methods involve the introduction of lubricants intended to protect the articular surface and lengthening of their half life and residence time by the introduction of anti-inflammatory medication and biologics.

The data described herein shows that such a treatment regimen preserves and augments lubrication of injured joints. Treatment of an inflamed rat joint following an ACL injury with a TNF-α inhibitor etanerept/ENBREL™ (Amgen) reestablishes some of the lubricin layer in the superficial zone. An increase in the appearance of lubricin was observed with ENBREL™ alone. Exogenous tribonectin is administered to protect the articular surface from wearing against one another. A combination of anti-inflammatory therapy in traumatized joints with a boundary lubricant (tribonectin) to restore chondroprotection re-establishes or increases lubricin concentration and decreases one or more symptoms of joint degeneration (e.g., pain, decreased mobility, decreased range of motion).

### Lubricin

Lubricin is a mucinous glycoprotein, secreted by synovial fibroblasts (Jay et al, J Ortho Res 2001, 19:677-687) and the superficial zone articular chondrocyte (Flannery CR et al, Biochem Biophys Res Commun 1999, 254:535-541) and is responsible for lubrication of articular surfaces. SF from patients with an acute ACL injury had decreased lubricin at early stage following injury (Elsaid K A et al, Trans Orthop Res Soc 2007; 32:160), which has been associated with increased cartilage damage.

Endogenous lubricin SF levels were significantly lowered following ACL injury, and were associated with increased cartilage damage. Furthermore, inflammatory cytokines e.g. IL-1β and TNF-α were shown to decrease lubricin synthesis (Jones ARC et al, Eur Cell Mater 2007; 31:40-45).

### Tribonectins

A tribonectin is an lubricating polypeptide, which contains at least one repeat of an amino acid sequence which is at least 50% identical to KEPAPTT (SEQ ID NO:3). For example, a tribonectin comprises a polypeptide, the amino acid sequence of which comprises at least one but less than 76 subunits, with each subunit comprising at least 7 amino acids and the amino acid sequence of each subunit being at least 50% identical to SEQ ID NO:3 with a non-identical amino acid being a conservative amino acid substitution. A tribonectin contains at least one O-linked lubricating moiety. Preferably the lubricating moiety is a β(1-3)Gal-GalNAc moiety. Optionally, the β(1-3)Gal-GalNAc is capped with NeuAc. The polypeptide contains an oligosaccharide, which is moiety is N-linked to an asparagine residue, e.g., the oligosaccharide is asialo-, digalactosylated bi-antennary, bisecting N-acteylglucosamine, tri-mannosyl core-fucosylated. Tribonectins are further described in U.S. Pat. Nos. 7,001,881; 6,960,562; 6,743,774; U.S. Patent Application Pub. No. 2004/0229804; and U.S. Patent Application Pub. No. 2004/0072741.

The amino acid sequence of the protein backbone of a lubricating polypeptide may differ depending on alternative splicing of exons of the human megakaryocyte stimulating factor (MSF) gene (GENBANK® accession number U70136). Tribonectins are purified naturally-occurring polypeptides or synthetically produced or recombinant polypeptides. In one example, the tribonectin has the amino acid sequence of a naturally-occurring full-length MSF; alternatively, the sequence corresponds to an alternative splice variant of the MSF gene. Optionally, the polypeptide compositions do not contain a lipid. For example, less than 10%, preferably less than 5%, and more preferably less than 1% of the lubricating polypeptide composition is a lipid. The polypeptide compositions are purified from fibronectin. For example, less than 10%, preferably less than 5%, and more preferably less than 1% of the lubricating polypeptide composition is a fibronectin.

The amino acid sequence of the backbone of purified, synthetic, or recombinant tribonectins is at least 50% identical to the amino acid sequence of a naturally-occurring tribonectin and possess at least 50% of the lubricating activity of a naturally-occurring tribonectin. A tribonectin lubricating polypeptide is a purified protein or polypeptide containing the amino acid sequence of human megakaryocyte stimulating factor (MSF) or a fragment thereof. For example, a tribonectin lubricating polypeptide contains amino acids 67-106 and 200-1140 of SEQ ID NO:1 and at least one O-linked oligosaccharide, but lacks amino acids 26-66 of SEQ ID NO:1. In other examples, the polypeptide further lacks amino acids 107-156 of SEQ ID NO:1 or amino acids 107-199 of SEQ ID NO:1, or the polypeptide contains amino acids 1-25, 67-106, and 200-1404 of SEQ ID NO:1. Alternatively, the polypeptide contains amino acids 67-106 and 200-1140 of SEQ ID NO:1 and at least one O-linked oligosaccharide, but lacks amino acids 107-156 of SEQ ID NO:1. In another variation, the lubricating polypeptide contains amino acids 67-106 and 200-1140 of SEQ ID NO:1 and at least one O-linked oligosaccharide, but lacks amino acids 157-199 of SEQ ID NO:1. For example, the polypeptide contains amino acids 1-156 and 200-1404 of SEQ ID NO:1 (lacking amino acids 157-199 of SEQ ID NO:1) or the polypeptide contains amino acids 1-106 and 200-1404 (lacking amino acids 107-199 of SEQ ID NO:1). The amino acid sequences are contiguous to one another or may be separated by intervening unrelated sequences. Preferably, the lubricating polypeptide contains residues encoded by exons 1, 3, and 6-12 of the MSF gene.

Polynucleotides encoding the polypeptide backbone of a lubricating polypeptide include nucleotides 232-351 and 631-3453 of SEQ ID NO:2 and lacks nucleotides 109-231 of SEQ ID NO:2. In another example, the polynucleotide contains nucleotides 232-351 and 631-3453 of SEQ ID NO:2 and lacks nucleotides 352-501 of SEQ ID NO:2. In yet another examples, the polynucleotide contains nucleotides 232-351 and 631-3453 of SEQ ID NO:2 and lacks nucleotides 502-630 of SEQ ID NO:2. A polynucleotide containing nucleotides 232-351 and 631-3453 of SEQ ID NO:2 and lacking nucleotides 352-630 of SEQ ID NO:2 is also within the invention as well as a polynucleotide containing nucleotides 232-351 and 631-3453 of SEQ ID NO:2 and lacking nucleotides 109-231 and 352-630 of SEQ ID NO:2. For example, the polynucleotide contains nucleotides 34-501 linked in-frame to nucleotides 631-4245 of SEQ ID NO:2 or nucleotides 34-501 linked in-frame to nucleotides 631-4245 of SEQ ID NO:2. The polynucleotide contains a first sequence containing nucleotides 34-501 of SEQ ID NO:2, a second sequence containing nucleotides 232-351 of SEQ ID NO:2, and a third sequence containing nucleotides 631-4245 of SEQ ID NO:2. The first, second and third sequences are linked in-frame. Preferably, the sequences described above are contiguous.

TABLE 1
MSF amino acid sequence

(SEQ ID NO:1)

(GENBANK ® U70136)
MAWKTLPIYLLLLLSVFVIQQVSSQDLSSCAGRCGEGYSRDATCNCDYNC
QHYMECCPDFKRVCTAELSCKGRCFESFERGRECDCDAQCKKYDKCCPDY
ESFCAEVHNPTSPPSSKKAPPPSGASQTIKSTTKRSPKPPNKKKTKKVIE
SEEITEEHSVSENQESSSSSSSSSSSSTIWKIKSSKNSAANRELQKKLKV
KDNKKNRTKKKPTPKPPVVDEAGSGLDNGDFKVTTPDTSTTQHNKVSTSP
KITTAKPINPRPSLPPNSDTSKETSLTVNKETTVETKETTTTNKQTSTDG
KEKTTSAKETQSIEKTSAKDLAPTSKVLAKPTPKAETTTKGPALTTPKEP
TPTTPKEPASTTPKEPTPTTIKSAPTTPKEPAPTTTKSAPTTPKEPAPTT
TKEPAPTTPKEPAPTTTKEPAPTTTKSAPTTPKEPAPTTPKKPAPTTPKE
PAPTTPKEPTPTTPKEPAPTTKEPAPTTPKEPAPTAPKKPAPTTPKEPAP
TTPKEPAPTTTKEPSPTTPKEPAPTTTKSAPTTTKEPAPTTTKSAPTTPK
EPSPTTTKEPAPTTPKEPAPTTPKKPAPTTPKEPAPTTPKEPAPTTTKKP
APTAPKEPAPTTPKETAPTTPKKLTPTTPEKLAPTTPEKPAPTTPEELAP
TTPEEPTPTTPEEPAPTTPKAAAPNTPKEPAPTTPKEPAPTTPKEPAPTT
PKETAPTTPKGTAPTTLKEPAPTTPKKPAPKELAPTTTKEPTSTTSDKPA
PTTPKGTAPTTPKEPAPTTPKEPAPTTPKGTAPTTLKEPAPTTPKKPAPK
ELAPTTTKGPTSTTSDKPAPTTPKETAPTTPKEPAPTTPKKPAPTTPETP
PPTTSEVSTPTTTKEPTTIHKSPDESTPELSAEPTPKALENSPKEPGVPT
TKTPAATKPEMTTTAKDKTTERDLRTTPETTTAAPKMTKETATTTEKTTE
SKITATTTQVTSTTTQDTTPFKITTLKTTTLAPKVTTTKKTITTTEIMNK
PEETAKPKDRATNSKATTPKPQKPTKAPKKPTSTKKPKTMPRVRKPKTTP
TPRKMTSTMPELNPTSRIAEAMLQTTTRPNQTPNSKLVEVNPKSEDAGGA
EGETPHMLLRPHVFMPEVTPDMDYLPRVPNQGIIINPMLSDETNICNGKP
VDGLTTLRNGTLVAFRGHYFWMLSPFSPPSPARRITEVWGIPSPIDTVFT
RCNCEGKTFFFKDSQYWRFTNDIKDAGYPKPIFKGFGGLTGQIVAALSTA
KYKNWPESVYFFKRGGSIQQYIYKQEPVQKCPGRRPALNYPVYGEMTQVR
RRRFERAIGPSQTHTIRIQYSPARLAYQDKGVLHNEVKVSILWRGLPNVV
TSAISLPNIRKPDGYDYYAFSKDQYYNIDVPSRTARAITTRSGQTLSKVW
YNCP

TABLE 2
MSF cDNA

(SEQ ID NO:2)

| | |
|---|---|
| 1 | gcggccgcga ctattcggta cctgaaaaca acgatggcat ggaaaacact tcccatttac |
| 61 | ctgttgttgc tgctgtctgt tttcgtgatt cagcaagttt catctcaaga tttatcaagc |
| 121 | tgtgcaggga gatgtgggga agggtattct agagatgcca cctgcaactg tgattataac |
| 181 | tgtcaacact acatggagtg ctgccctgat ttcaagagag tctgcactgc ggagctttcc |
| 241 | tgtaaaggcc gctgctttga gtccttcgag agagggaggg agtgtgactg cgacgcccaa |
| 301 | tgtaagaagt atgacaagtg ctgtcccgat tatgagagtt tctgtgcaga agtgcataat |
| 361 | cccacatcac caccatcttc aaagaaagca cctccacctt caggagcatc tcaaaccatc |
| 421 | aaatcaacaa ccaaacgttc acccaaacca ccaaacaaga agaagactaa gaaagttata |
| 481 | gaatcagagg aaataacaga agaacattct gtttctgaaa atcaagagtc ctcctcctcc |
| 541 | tcctcctctt cctcttcttc ttcaacaatt tggaaaatca agttttccaa aaattcagct |
| | EXON 6 |
| 601 | gctaatagag aattacagaa gaaactcaaa gtaaaagata acaagaagaa cagaactaaa |
| 661 | aagaaaccta cccccaaacc accagttgta gatgaagctg gaagtggatt ggacaatggt |
| 721 | gacttcaagg tcacaactcc tgacacgtct accacccaac acaataaagt cagcacatct |
| 781 | cccaagatca caacagcaaa accaataaat cccagaccca gtcttccacc taattctgat |
| 841 | acatctaaag agacgtcttt gacagtgaat aaagagacaa cagttgaaac taaagaaact |
| 901 | actacaacaa ataaacagac ttcaactgat ggaaaagaga agactacttc cgctaaagag |
| 961 | acacaaagta tagagaaaac atctgctaaa gatttagcac ccacatctaa agtgctggct |
| 1021 | aaacctacac ccaaagctga aactacaacc aaaggccctg ctctcaccac tcccaaggag |
| 1081 | cccacgccca ccactcccaa ggagcctgca tctaccacac ccaaagagcc cacacctacc |
| 1141 | accatcaagt ctgcacccac cacccccaag gagcctgcac ccaccaccac caagtctgca |
| 1201 | cccaccactc ccaaggagcc tgaacccacc accaccaagg agcctgcacc caccactccc |
| 1261 | aaggagcctg cacccaccac caccaaggag cctgcaccca ccaccaccaa gtctgcaccc |
| 1321 | accactccca aggagcctgc acccaccacc cccaagaagc ctgccccaac tacccccaag |
| 1381 | gagcctgcac ccaccactcc caaggagcgc acacccacca ctcccaagga gcctgcaccc |
| 1441 | accaccaagg agcctgcacc caccactccc aaagagcttg cacccactgc ccccaagaag |
| 1501 | cctgccccaa ctacccccaa ggagcctgca cccaccactc ccaaggagcc tgcacccacc |
| 1561 | accaccaagg agccttcacc caccactccc aaggagcctg cacccaccac caccaagctc |
| 1621 | gcacccacca ctaccaagga gcctgcaccc accactacca agtctgcacc caccactccc |
| 1681 | aaggagcctt cacccaccac caccaaggag cctgcaccca ccactcccaa ggagcctgca |
| 1741 | cccaccaccc ccaagaagcc tgccccaact acccccaagg agcctgcacc caccactccc |
| 1801 | aaggaacctg cacccaccac caccaagaag cctgcagcca ccgctcccaa agagcctgcc |
| 1861 | ccaactaccc ccaaggagac tgcacccacc acccccaaga agctcacgcc caccaccccc |
| 1921 | gagaagctcg cacccaccac ccctgagaag cccgcaccca ccacccctga ggagctcgca |
| 1981 | cccaccaccc ctgaggagcc cacacccacc acccctgagg agcctgctcc caccactccc |
| 2041 | aaggcagcgg ctcccaacac ccctaaggag cctgctccaa ctacccctaa ggagcctgct |
| 2101 | ccaactaccc ctaaggagcc tgctccaact acccctaagg agactgctcc aactacccct |
| 2161 | aaagggactg ctccaactac cctcaaggaa cctgcaccca ctactcccaa gaagcctgcc |
| 2221 | tccaaggagc ttgcacccac caccaccaag gagcccacat ccaccacctc tgacaagccc |
| 2281 | gctccaacta cccctaaggg gactgctcca actaccccta adgadcctgc tccaactacc |
| 2341 | cctaaggagc ctgctccaac tacccctaag gggactgctc caactaccct caaggaacct |
| 2401 | gcacccacta ctcccaagaa gcctgccccc aaggagcttg cacccaccac caccaagggg |
| 2461 | cccacatcca ccacctctga caagcctgct ccaactacac ctaaggagac tgctccaact |
| 2521 | acccccaagg agcctgcacc cactaccccc aagaagcctg ctccaactac tcctgagaca |
| 2581 | cctcctccaa ccacttcaga ggtctctact ccaactacca ccaaggagcc taccactatc |
| 2641 | cacaaaagcc ctgatgaatc aactcctgag ctttctgcag aacccacacc aaaagctctt |
| 2701 | gaaaacagtc ccaaggaacc tggtgtacct acaactaaga ctcctgcagc gactaaacct |
| 2761 | gaaatgacta caacagctaa agacaagaca acagaaagag acttacgtac tacacctgaa |
| 2821 | actacaactg ctgcacctaa gatgacaaaa gagacagcaa ctacaacaga aaaaactacc |
| 2881 | gaatccaaaa taacagctac aaccacacaa gtaacatcta ccacaactca agataccaca |
| 2941 | ccattcaaaa ttactactct taaaacaact actcttgcac ccaaagtaac tacaacaaaa |
| 3001 | aagacaatta ctaccactga gattatgaac aaacctgaag aaacagctaa accaaaagac |
| 3061 | agagctacta attctaaagc gacaactcct aaacctcaaa agccaaccaa agcacccaaa |
| 3121 | aaacccactt ctaccaaaaa gccaaaaaca atgctcagag tgagaaaacc aaagacgaca |
| 3181 | ccaactcccc gcaagatgac atcaacaatg ccagaattga aaccctacctc aagaatagca |
| 3241 | gaagccatgc tccaaaccac caccagacct aaccaaactc caaactccaa actagttgaa |
| 3301 | gtaaatccaa agagtgaaga tgcaggtggt gctgaaggag aaacacctca tatgcttctc |
| 3361 | aggccccatg tgttcatgcc tgaagttact cccgacatgg attacttacc gagagtaccc |
| 3421 | aatcaaggca ttatcatcaa tcccatgctt tccgatgaga ccaatatatg ccatggtaag |
| 3481 | ccagtagatg gactgactac tttgcgcaat gggacattag ttgcattccg aggtcattat |
| 3541 | ttctggatgc taagtccatt cagtccacca tctccagctc gcagaattac tgaagtttgg |
| 3601 | ggtattcctt cccccattga tactgttttt actaggtgca actgtgaagg aaaaactttc |
| 3661 | ttctttaagg attctcagta ctggcgtttt accaatgata taaaagatgc agggtacccc |
| 3721 | aaaccaattt tcaaaggatt tggaggacta actggacaaa tagtggcagc gctttcaaca |
| 3781 | gctaaatata agaactggcc tgaatctgtg tattttttca agagaggtgg cagcattcag |
| 3841 | cagtatattt ataaacagga acctgtacag aagtgccctg gaagaaggcc tgctctaaat |
| 3901 | tatccagtgt atggagaaat gacacaggtt aggagacgtc gctttgaacg tgctatagga |
| 3961 | ccttctcaaa cacacaccat cagaattcaa tattcacctg ccagactggc ttatcaagac |
| 4021 | aaaggtgtcc ttcataatga agttaaagtg agtatactgt ggagaggact tccaaatgtg |
| 4081 | gttacctcag ctatatcact gcccaacatc agaaaacctg acggctatga ttactatgcc |
| 4141 | ttttctaaag atcaatacta taacattgat gtgcctagta gaacagcaag agcaattact |
| 4201 | actcgttctg ggcagacctt atccaaagtc tggtacaact gtccttagac tgatgagcaa |
| 4261 | aggaggagtc aactaatgaa gaaatgaata ataaattttg acactgaaaa acattttatt |
| 4321 | aataaagaat attgacatga gtataccagt ttatatataa aaatgttttt aaacttgaca |
| 4381 | atcattacac taaaacagat ttgataatct tattcacagt tgttattgtt tacagaccat |
| 4441 | ttaattaata tttcctctgt ttattcctcc tctccctccc attgcatggc tcacacctgt |
| 4501 | aaaagaaaaa agaatcaaat tgaatatatc ttttaagaat tcaaaactag tgtattcact |
| 4561 | taccctagtt cattataaaa aatatctagg cattgtggat ataaaactgt tgggtattct |
| 4621 | acaacttcaa tggaaattat tacaagcaga ttaatccctc tttttgtgac acaagtacaa |
| 4681 | tctaaaagtt atattggaaa acatggaaat attaaaattt tacactttta ctagctaaaa |
| 4741 | cataatcaca aagctttatc gtgttgtata aaaaaattaa caatataatg gcaataggta |
| 4801 | gagatacaac aaatgaatat aacactataa cacttcatat tttccaaatc ttaatttgga |
| 4861 | tttaaggaag aaatcaataa atataaaata taagcacata tttattatat atctaaggta |
| 4921 | tacaaatctg tctacatgaa gtttacagat tggtaaatat catctgctca acatgtaatt |
| 4981 | atttaataaa actttggaac attaaaaaaa taaattggag gcttaaaaaa aaaaaaaaaa |
| 5041 | a |

In addition to the tribonectins described above, recombinant lubricin molecules and other compositions described in U.S. Patent Application Pub. No. 2007/0191268 are used in combination with tribonectins, HA, and/or inhibitors of proinflammatory mediators to preserve, prolong, or augment joint lubrication.

### p38 and Inflammation

A strong link has been established between the p38 pathway and inflammation. Inhibition of inflammation via the p38 pathway leads to enhanced lubricin/tribonectin production. The activation of the p38 pathway plays essential roles in the production of proinflammatory cytokines (IL-1, TNF- and IL-6); induction of enzymes such as COX-2 which controls connective tissue remodeling in pathological conditions; expression of intracellular enzymes such as iNOS, a regulator of oxidation; induction of VCAM-1 and other adherent proteins along with other inflammatory related molecules. In addition, a regulatory role for p38 in the proliferation and differentiation of immune system cells such as GM-CSF, EPO, CSF and CD-40 has been established. Inhibition of elements of this pathway together with administration of a tribonectin lead to improved joint lubrication over prolonged periods of time.

### Inhibitors of TNF-Alpha

Exemplary functional blockers of TNF-α include, but are not limited to, recombinant and/or soluble TNFα receptors, monoclonal antibodies, and small molecule antagonists and/or inverse agonists. Exemplary commercial TNF-α blocking agents include, etanerept/ENBREL™, infliximab/Remicade, and adalimumab/Humira.

Etanercept (ENBREL™, co-marketed by Amgen and Wyeth) is a recombinant human soluble TNF-α receptor (DrugBank BTD0052). It is a small protein (75 kDa) that binds TNFα and decreases its role in inflammation. ENBREL™ is a dimeric fusion protein comprised of the extracellular ligand-binding portion of the human 75 kilodalton (p75) tumor necrosis factor receptor (TNFR) linked to the Fc portion of human IgG1 . The Fc component of etanercept contains the CH2 domain, the CH3 domain and hinge region, but not the CH1 domain of IgG1. Etanercept is produced by recombinant DNA technology in a Chinese hamster ovary (CHO) mammalian cell expression system and is comprised of 934 amino acids (GenBank M32315). Etanercept binds specifically to tumor necrosis factor (TNF) and blocks its interaction with cell surface TNF receptors. The biological activity of TNF is dependent upon binding to either cell surface receptor (p75 or p55). Etanercept is a dimeric soluble form of the p75 TNF receptor that can bind to two TNF molecules, thereby effectively removing them from circulation.

### Inhibitors of IL-1

Inhibitors or antagonists of proinflammatory cytokine IL-1 include anakinra/KINERET™ (recombinant human IL-1Ra, rhIL-1Ra). IL-1Ra is an endogenous receptor antagonist, which is primarily produced by activated monocytes and tissue macrophages and inhibits the activities of the proinflammatory forms of IL-1 by competitively binding to IL-1 receptor. IL-1Ra is an inducible gene that is often upregulated in inflammatory conditions. Although the binding affinity of natural IL-1Ra is similar to that of IL-1, it lacks IL-1 agonist activity.

Exemplary IL-1Ra compositions include NCBI Accession No. NM_{—}173842 (Human IL-1Ra, transcript 1), which encodes amino acid sequence NCBI Accession No. NM_{—}173842; Human IL-1Ra, transcript 2 NCBI Accession No. NM_{—}173841 which encodes amino acid sequence NCBI Accession No. NM_{—}173841; and Human IL-1Ra, transcript 3 NCBI Accession No. NM_{—}000577 which encodes amino acid sequence (NCBI Accession No. NM_{—}000577). Anakinra/KINERET™ is encoded by transcript sequence NCBI Accession No. M55646, and the amino acid sequence of Anakinra/KINERET™ is recorded at DrugBank Accession No. BTD00060.

Inhibitory antibodies include anti-IL-1α (U.S. Patent No. 20030026806), anti-IL-1β (U.S. Patent No.20030026806) and humanized monoclonal anti-IL-1R (Amgen, U.S. Patent No. 2004022718 and Roche, U.S. Patent No. 2005023872).

### EXAMPLE 1

### Reversible Rise in Joint Ex-Vivo μ is a Result of Decreased Synovial Lubricin Expression in an Antigen-Induced Arthritis Model

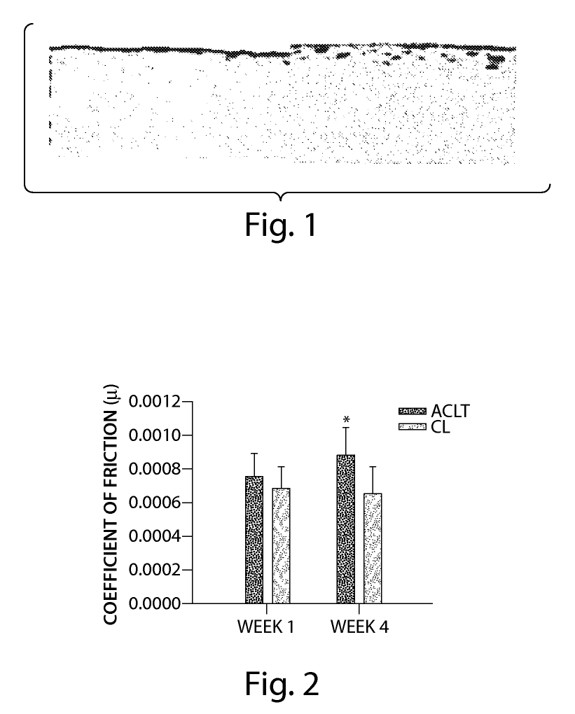

Data obtained with an antigen-induced arthritis rat model using limbs studied ex vivo showed that a reversible rise in joint ex-vivo μ was attributable to decreased synoviocyte lubricin expression. The ex-vivo μ of arthritic joints was significantly (P<0.001) higher than the μ of contra-lateral joints at 7, 21 and 24 days following the induction of arthritis. Lubricin mRNA expression in the synovial tissues of the arthritic joints relative to those of the contralateral joints was significantly (P<0.001) lower at days 4 and 7 compared to control. At day 28 following arthritis induction, there was no significant difference between relative lubricin mRNA expression in the arthritic and control joints. Decreased lubricin expression in the superficial zone of articular chondrocytes was demonstrated by decreased lubricin staining in the arthritic joints at 7 days following the initiation of arthritis (FIG. 1).

### EXAMPLE 2

### ACL Injury Results in Loss of Joint Lubricating Ability and Damage to the Articular Surface in Rats

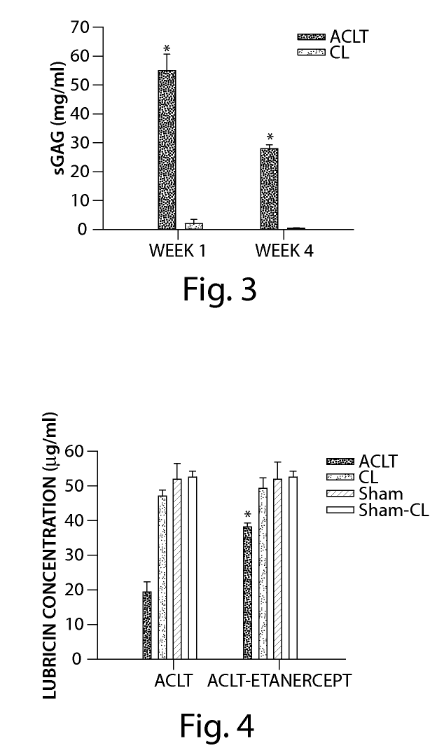

The method described above was applied to a rat ACL injury model. 12 rats underwent ACL transection (ACLT) of the right rear limb knee joints. Following 7 and 28 days both the right and left rear limbs from 6 animals at each time point were harvested and underwent pendulum studies to determine ex-vivo μ. Following pendulum studies, joints were lavaged and SF lavages were analyzed for sulfated glycosaminoglycan (sGAG) levels using alcian blue binding assay. Immunohistochemical staining for surface damage and presence of lubricin, using a lubricin-specific monoclonal antibody was also performed. The ex-vivo μ values of ACLT joints were significantly elevated at 4 weeks following injury compared to either contralateral (CL) joints at week 4 or ACLT joints at 1 week following the injury (FIG. 2). This time-dependent elevation of friction was associated with increased sGAG SF lavage levels (FIG. 3). At 1 week following the injury, sGAG levels dramatically increased in the ACLT joints compared to the contra-lateral joints. At 4 weeks following the injury, the ACLT joints demonstrated elevated SF lavage sGAG levels compared to the contralateral joints. However, sGAG levels of SF lavages at week 4 were significantly (p<0.001) lower than sGAG levels of SF lavages at week 1.

### EXAMPLE 3

### Administration of Etanercept Blunts the Catabolic Decrease in Synovial Fluid Lubricin Concentration Following ACLT

Studies were carried out to investigate the effects of blocking TNF-α at an early stage after injury on SF lubricin levels, joint lubrication and cartilage damage. Inhibition of TNF-α was performed using etanercept, a TNF-α soluble receptor that is approved for treatment of rheumatoid arthritis.

The following reagents and methods were used to evaluate inhibition of the effects of TNF-α by etanercept and the preservation of joint lubrication and decrease cartilage damage following an ACL injury. ACL-transection (ACLT) model and entanercept administration: Male Lewis rats 7-8 weeks were assigned to either an ACL-transection group (n=5), etanercept-treated ACL-transection group (n=5) or a sham-group (n=4). Etanercept was given subcutaneously at 0.5 mg/kg on days 1, 3, 5, 7, 9, 11, and 13 following ACLT. At 14 days following surgery, ACLT, contralateral (CL) and sham (S) joints were harvested and lavaged. Lavaging was performed by injecting 100 μl in the joint capsule, flexing and extending the joint for ten times and aspirating 50 μl of fluid. SF Lubricin levels: Lubricin levels were determined using sandwich ELISA employing a lubricin-specific monoclonal antibody, and peanut agglutinin (PNA). Ex-vivo joint μ: Articular joints' μ values were determined using modified Stanton pendulum (Elsaid K A et al, Arthritis Rheum 2007; 56:108-116). sGAG levels in SF lavages: Total sGAG levels were determined by Alcian blue binding assay.

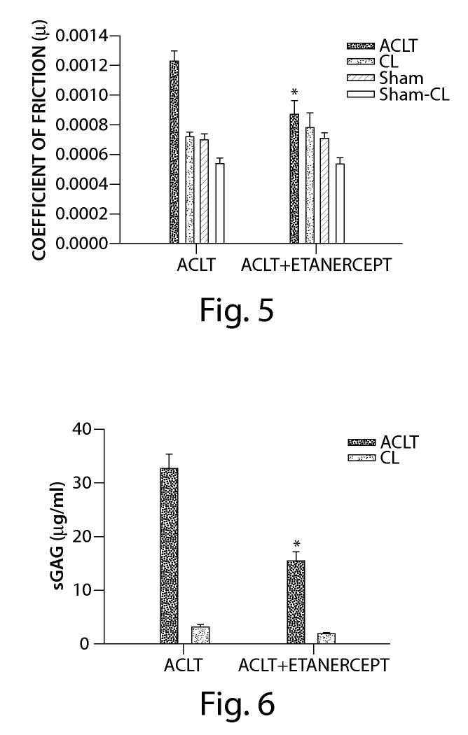

The concentrations of lubricin in the SF lavages were found to be significantly (p<0.001) higher in the etanercept-treated ACLT joints compared to the non-treated joints (FIG. 4). The ex-vivo joints μ valves were significantly (p=0.021) lower in the etanercept-treated ACLT compared to the non-treated joints (FIG. 5). sGAG levels in SF were significantly (p<0.001) lower in the etanercept-treated group compared to the non-treated group (FIG. 6). At an early stage following an acute ACL injury, inhibition of the effects of TNF-α leads to a significant increase in SF lubricin levels and decrease in joint μ. This effect is associated with decreased cartilage damage as determined by a decrease in sGAG turnover. Early intervention following ACL injury by blocking the effects of TNF-α preserved articular joint's chondroprotective mechanisms and decreased the extent of cartilage-injury.

ENBREL™ administered subcutaneously led to an upregulation of lubricin, leading to effective chondroprotection and prevention/reduction of cartilage wear. The data indicate that a TNF-α inhibitor (e.g., ENBREL™) is effective in increasing lubricin levels. A combination of recombinant lubricin and ENBREL™ leads to a greater clinical response in the cartilage preservation endpoint by the inhibition of wear. Early intervention is beneficial in retarding long-term cartilage damage and development of secondary osteoarthritis following ACL injury.

### EXAMPLE 4

### Comparison of Early and Late Inhibition of TNF-α in Restoring Chondroprotection by Lubricin in the ACL Transection Injury Model

Etanercept was administered to animals, and condroprotection was evaluated using an ACL-transection model. Male Lewis rats 7-8 weeks were assigned to either an ACL-transection group (n=5), 2 etanercept-treated ACLT groups (n=12, 6 in each group) or a sham surgery group (n=4). Etanercept was given subcutaneously at 0.5 mg/kg on days 8, and 15 following transection (Treatment A) or on days 15 and 22 (Treatment B). At 28 days following transection, ACL-transected (ACLT), contralateral (CL) and sham joints were harvested and lavaged. Lavaging was performed by injecting 50 μl of normal saline in the joint capsule, flexing and extending the joint for ten times and then aspirating 30 μl of fluid.

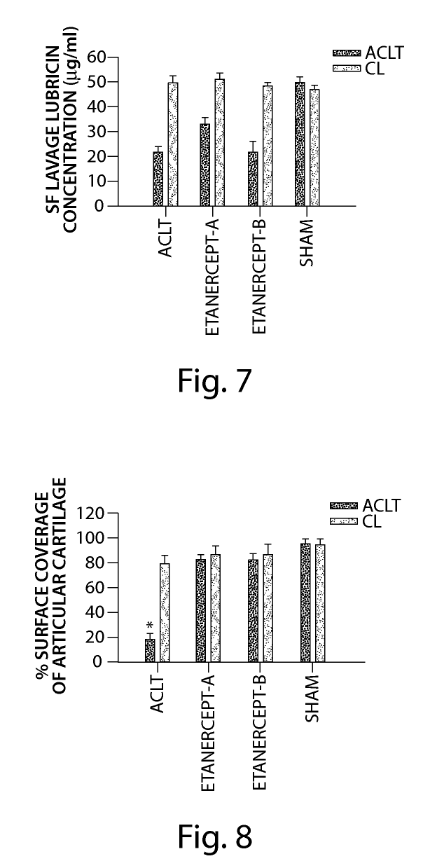

SF lubricin levels were determined using a sandwich ELISA employing a lubricin-specific monoclonal antibody such as 9G3, and peanut agglutinin. Surface-associated lubricin coverage was calculated from 9G3-stained histological specimens. Quantitiation of lubricin staining intensity was calculated using Image Pro-Plus software (Media Cybernetics, MD, USA) with pre-determined threshold parameters and expressed as percentage of lubricin surface coverage. An average of lubricin surface coverage of the medial and lateral regions of femur and tibial cartilage surfaces was calculated. Articular joints' μ values were determined ex vivo using modified Stanton pendulum (Elsaid K A et al., Arthritis Rheum., 2007,56:108-116).The SF lavage lubricin levels in treatment-A ACLT joints were found to be significantly (p<0.01) higher than in control or treatment B joints (FIG. 7). SF lavage lubricin levels in CL joints were comparable across the different groups. Lubricin coverage of articular surfaces was significantly (p<0.01) lower in the ACLT joints compared to treatments A, B and sham surgery. Lubricin coverage in the CL joints was comparable across the different groups. The joints' μ values were comparable in treatments A, and B and were lower than the μ values of ACLT joints, but approximated the μ values of sham surgery joints (Table 3).

TABLE 3

| | ACLT | Treatment A | Treatment B | Sham |
|---|---|---|---|---|
| ACLT | 8.9*10⁻⁴ ± | 6.5*10⁻⁴ ± | 6.6*10⁻⁴ ± | 6.1*10⁻⁴ ± |
| | 4.6*10⁻⁵ | 6.1*10⁻⁵ | 8.6*10⁻⁵ | 1.6*10⁻⁵ |
| CL | 6.6*10⁻⁴ ± | 4.7*10⁻⁴ ± | 4.9*10⁻⁴ ± | 6.2*10⁻⁴ ± |
| | 1.5*10⁻⁵ | 1.5*10⁻⁵ | 8.6*10⁻⁵ | 1.6*10⁻⁵ |

Table 3 shows the mean coefficient of friction (μ)±standard error of the mean (SEM) of ACLT and CL joints of different groups. Inhibition of the effects of TNF-α by etanercept treatment re-establishes lubricin coverage on the surface of articular cartilage and is associated with a reduction in joints' coefficient of friction compared to non-treated joints. Initiating TNF-α inhibition early (treatment A) results in higher SF lavage lubricin levels compared to later intervention (treatment B). Differences in SF lavage lubricin levels did not correspond to changes in surface-associated lubricin or changes in ex-vivo μ values. Articular cartilage surface coverage with lubricin was associated with a lowering of μ values in etanercept-treated joints compared to no treatment. These results indicate that surface-associated lubricin is more important than SF lubricin in modulating articular lubrication ability. Two early administrations of etanercept was found to be more effective than later treatment in re-establishing high SF lubricin which may serve as a reservoir of lubricin for articular surfaces. This administration approach is useful as an alternative or adjunct to intra-articular lubricin supplementation. TNF-α inhibitory compositions either alone or in combination with a tribonectin confer a clinical benefit by increasing the amount of surface-associated lubricin and reducing the co-efficient of friction in the joint.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for preserving or augmenting joint lubrication, comprising contacting a tissue of said joint with a tribonectin and an inhibitor of a proinflammatory mediator.

2. The method of claim 1, wherein said mediator is a cytokine.

3. The method of claim 2, wherein said cytokine is interleukin-1β (IL-1β) or tumor necrosis factor-α (TNF-α).

4. The method of claim 1, wherein said inhibitor reduces the expression or function of a proinflammatory IL-1.

5. The method of claim 1, wherein said inhibitor is IL-1RA

6. The method of claim 1, wherein said inhibitor reduces the expression or function of TNF-α.

7. The method of claim 1, wherein said inhibitor is an antibody that bind to TNF-α.

8. The method of claim 1, wherein said inhibitor is ENBREL™.

9. The method of claim 1, wherein said inhibiter is administered directly into a joint.

10. The method of claim 1, wherein said inhibitor is administered subcutaneously.

11. The method of claim 1, wherein said tribonectin is administered directly into a joint.

12. The method of claim 1, wherein said tribonectin and said inhibitor are administered simultaneously.

13. The method of claim 1, wherein said tribonectin and said inhibitor are administered sequentially.

14. A composition comprising a tribonectin and an inhibitor of a proinflammatory mediator.

15. The composition of claim 14, further comprising hyaluronic acid.
